(19) United States
(12) Plant Patent Application Publication
(10) Pub. No.: US 2005/0193453 P1
(43) Pub. Date: Sep. 1, 2005

(54) Variety of Daylily plant named 'BERSTEL'

(50) HEMEROCALLIS HYBRIDA; DAYLILY

(76) Inventor: Leo Berbee, AD Lisse (NL)

Correspondence Address: FULWIDER PATTON LEE & UTECHT, LLP, 200 OCEANGATE, SUITE 1550, LONG BEACH, CA, 90802, US

(21) Appl. No.: 10/789,483

(22) Filed: Feb. 27, 2004

(51) Int. Cl. A01H 5/00

(52) U.S. Cl. PLT/312

(57) ABSTRACT

A new and distinct cultivar of Daylily plant named ‘BERSTEL’ characterized by its distinctive lemon-chiffon yellow flowers and strong citrus fragrance with blooming periods lasting from late spring/early summer through first frost.

### BACKGROUND OF THE NEW PLANT

The present invention relates to a new and distinct cultivar of Daylily, botanically known as Daylily hemerocallis hybrid and further known by the varietal name ‘BERSTEL’. This new variety is also sold under the trade name STELLA SUPREME (Breeder's Ref. No. D94-17). The new variety was discovered in a seedling selection program by the inventor, Leo Berbee, in Lisse, The Netherlands in 1994. The parental cultivar are unknown. The cultivar was isolated by the inventor, Leo Berbee, at Lisse, the Netherlands, by seedling selection primarily on the basis of ornamental appeal, particularly flower color.

Following selection of the seedling the cultivar was propagated vegetatively from clump divisions at Lisse, The Netherlands in 1994. Continued observation of the vegetative divisions resulting from asexual reproduction of clump divisions taken at Lisse, The Netherlands having confirmed that the distinguishing features of this new cultivar came true, remain stable and are retained through successive propagation.

### SUMMARY OF THE INVENTION

Plants of the cultivar ‘BERSTEL’ have not been observed under all possible environmental and cultural conditions. The phenotype may vary somewhat with variations in environment such as temperature, light intensity, length of day, irrigation amount and frequency, and/or fertilizer rate without, however, any variance in genotype.

The following traits have been repeatedly observed and are determined to be among the unique characteristics of ‘BERSTEL’. These characteristics distinguish ‘BERSTEL’ as a new and distinct cultivar:

1. Distinctive lemon-chiffon yellow flower color; and

2. Long blooming season.

Plants of the new Daylily can be compared to plants of the Daylily cultivar ‘Stella d'Oro’ (unpatented). Compared to plants of the cultivar ‘Stella d'Oro’, the new Daylily has a distinctive generally triangular shaped flower with a lemon-chiffon yellow in contrast to the rounded golden-yellow flower of the comparator. The new Daylily also has a longer blooming season than the comparator.

### BRIEF DESCRIPTION OF THE DRAWINGS

The invention is depicted in the accompanying colored photographs in which:

FIG. 1 shows a close up of the flower;

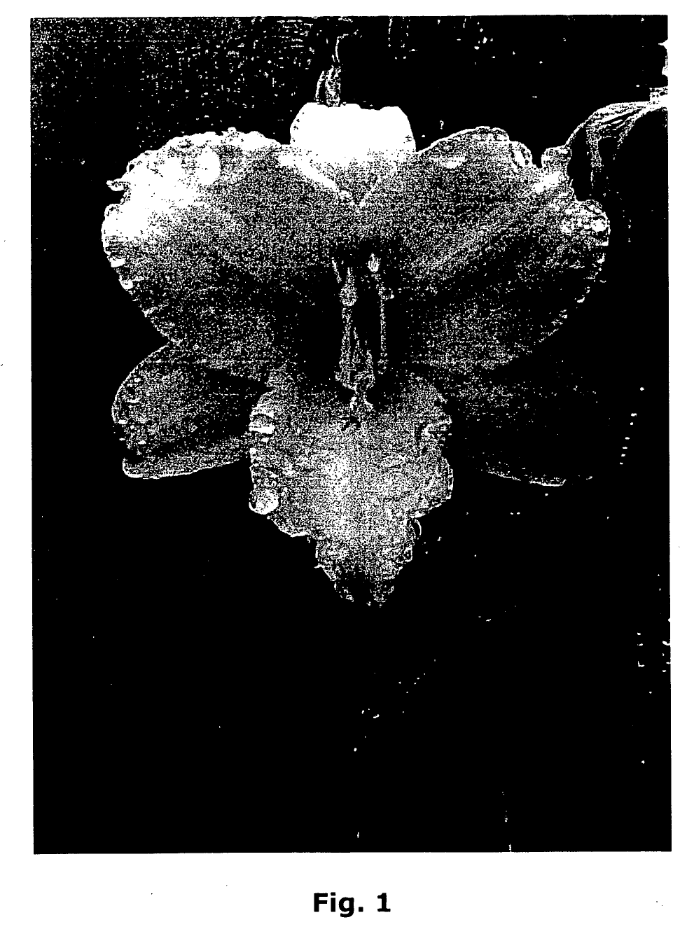

FIG. 2 shows the plant as a clump growing in a garden environment; and

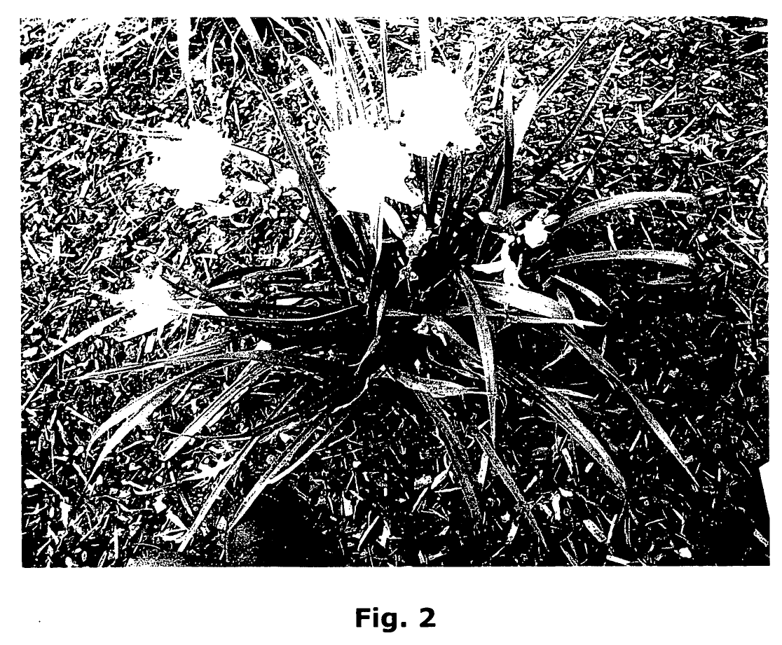

FIG. 3 is a side-by-side comparison of the new Daylily on the left with the comparator ‘Stella d'Oro’.

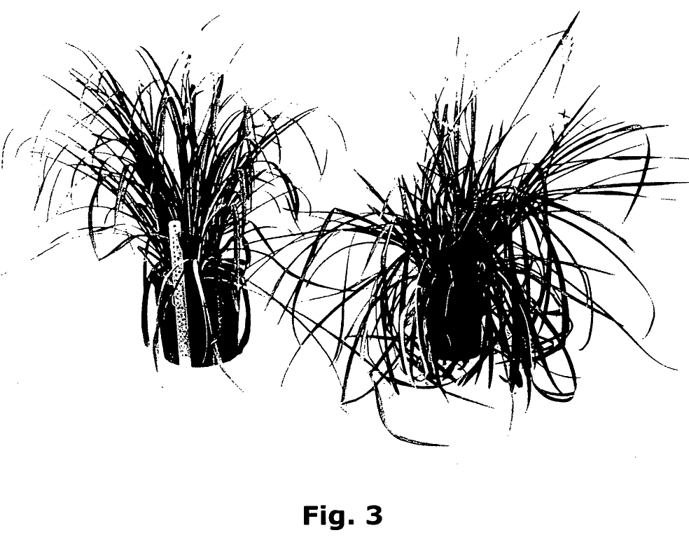

The photographs depict the overall appearance of the new Daylily, showing the colors as true as it is reasonably possible to obtain in colored reproductions of this type. Colors in the photographs may differ slightly from the color values cited in the Detailed Botanical Description which more accurately describe the actual colors of the new Daylily.

### DETAILED BOTANICAL DESCRIPTION OF THE NEW PLANT

The following detailed description sets forth characteristics of the new cultivar. The data which defines each characteristic was collected from asexual reproductions grown in an open greenhouse with irrigation provided by drip feeding in a hydroponic system, and carried out by the breeder at Clyde, Victoria, Australia, approximately 38 degrees, 9 minutes South latitude at 16 m elevation. The plant histories of 12 plants were taken on liners, potted into 150 mm pots of a pine bark potting mix, sifting on tray channels, in approximately September 2003.

In the following table of comparative features, color references are made to The Royal Horticultural Society of London Colour Chart, 1995 Edition. The color values were determined on plant material grown in an open greenhouse at Clyde, Victoria, Australia.

| | ‘BERSTEL’ | ‘Stella d'Oro’ |
|---|---|---|
| The Plant | | |
| Plant Growth Rate | Moderately vigorous | Moderately |
| | | vigorous |
| Length of Flowering Season | Remontant | |
| Time of Flowering Season | Early Spring to Late | |
| | Summer | |
| Density | Thick | |
| Growth Type | Clumping | |
| Height | Low | |
| Width | Medium | |
| Leaf | | |
| Color-Main | 146B | 137B |
| Characteristics | Basal, linear, tapered, | |
| | falcate | |
| Mean Length (mm) | 59.75 | 45.6 |
| Mean Width (mm) | 14.69 | 12.09 |
| Apex | Pointed | |
| Margin | Flat | |
| Texture | Smooth | |
| Shape | Flattened Convex | |
| Stem | Sessile | |
| Scapes | | |
| Branching arrangement | Taller than foliage, | |
| | forked but sparsely | |
| | branched | |
| Mean stem diameter (mm) | 7.3 | 7.87 |
| (just below inflorescence) | | |
| INFLORESCENCE | | |
| Length of flowering period | Remontant | |
| Number of florets | 10-20 | |
| Mean floret length (mm) | 74.93 | 71.57 |
| Lastingness of bloom | 1 day | |
| Fragrance of bloom | Strong, citrus | |
| | fragrance | |
| Diameter | Small | |
| Mean Length (mm) | 84.5 | 69.5 |
| Form front | Triangular | |
| Form profile | Recurved | |
| Color type | Self | |
| Color Midzone outside | 4C | 16A-16B |
| Color Margin outside | 4C | 21C |
| Color Midzone inside | 5B | 21B |
| Color Margin inside | 5C | 21C |
| Flower bud | | |
| Shape | Long Ovate | |
| Mean Length (mm) (at time | 53 | 52 |
| of sepal separation) | | |
| Mean width (mm) (at time | 14 | 12 |
| of sepal separation) | | |
| Color | 150B | 145A |
| Petal | | |
| Number | 6 | |
| Mean length (mm) (as at | 57.35 | 51.08 |
| separation) | | |
| Mean width (mm) | 29.14 | 20.015 |
| Arrangement | 3 inner, 3 outer arranged | |
| | in opposition. | |
| Pedicel | | |
| type | Bracteate, borne in a | |
| | close raceme | |
| Color | 137C | 137D |
| Prickles | Absent | |
| Mean Length (mm) | 2.73 | 1.69 |
| Sepals | | |
| Number | Nil | |
| Basal Spot | | |
| Presence | Absent | |
| Stamen | | |
| Filament color | 17C | 17A |
| Mean filament length (mm) | 7 | 6 |
| Anther color | 10D | 14D |
| Mean anther length (mm) | 41.5 | 35.5 |
| (base to bend) | | |
| Pistils | | |
| Mean style length (mm) | 59.4 | 51.2 |
| Style color | 10C | 16B |
| Seed vessel | | |
| Fruit Presence | 3 valved, few seeded, | |
| | locule capsule | |

### General Characteristics

‘BERSTEL’ is primarily distinguished from plants of the Daylily cultivar ‘Stella d'Oro’ and other hybrid cultivars of the Daylily genus by its uniquely distinctive lemon-chiffon yellow colored flowers giving off a strong citrus fragrance and projecting from scapes reaching about 20 inches (508 mm). ‘BERSTEL’ is an ever-blooming plant beginning its blooming season in late spring/early summer and continuing until first frost with a consistent array of flowers throughout the season. The foliage includes long flattened leaves of a medium green color lighter than the comparator.

1. A new and distinct variety of Daylily plant, substantially as shown and described.
